(19) United States
(12) Patent Application Publication
(10) Pub. No.: US 2023/0358723 A1
(43) Pub. Date: Nov. 9, 2023

(54) TRACKING HIDE AND LEATHER IN A SUPPLY CHAIN PROCESS

(71) Applicant: SECURITY MATTERS LTD., D.N. Hevel Eilot (IL)

(72) Inventors: Yifat BAREKET, Reut (IL); Michal FIRSTENBERG, Rehovot (IL); Hagit SADE, Ramat-Gan (IL); Zeren BROWNE, Wembley (AU); Tehila NAHUM, Holon (IL); Nataly TAL, Nes Ziona (IL); Mor KAPLINSKY, Herzelia (IL); Dana GASPAR, Tel Aviv (IL); Haggai ALON, Kibbutz Naan (IL); Ron DAFNI, Tel Aviv (IL); Chen NACHMIAS, Tel Aviv (IL); Avital TRACHTMAN, Tel Aviv (IL); Maria CHUCHAEV, Kibbutz Yahel (IL); Nadav YORAN, Tel Aviv (IL); Michal BURCK ZALTZMAN, Tel Aviv (IL)

(21) Appl. No.: 18/040,312

(22) PCT Filed: Aug. 3, 2021
(86) PCT No.: PCT/IL2021/050939

§ 371 Date: Feb. 2, 2023

Related U.S. Application Data
(60) Provisional application No. 63/060,346, filed on Aug. 3, 2020.
(60) Provisional application No. 63/069,427, filed on Aug. 24, 2020.

(51) Int. Cl. G01N 33/44; C14B 17/00; G01N 23/223; D06H 1/00; C14C 1/04

(52) U.S. Cl. CPC G01N 33/447; C14B 17/005; G01N 23/223; D06H 1/00; C14C 1/04; G01N 2223/076

(57) ABSTRACT

A process is provided for marking leather manufactured from hide with an XRF-identifiable marker, the process comprising treating a hide or processed leather with a formulation comprising at least one XRF-identifiable marker to embed said marker in the hide or processed leather, to thereby obtain a marked hide or marked leather, wherein the XRF-identifiable marker is not a native material to a hide or leather or involved in a process for its manufacturing.

### TECHNOLOGICAL FIELD

The technology disclosed herein generally concerns means for tracking hide and leather, as well as finished leather products in supply chain production of hide and leather.

### BACKGROUND

The production of leather and leather products from hide is a complex multi-stage process which involves a plurality of chemical steps starting from the slaughterhouse via the tannery and the production chain to the distributor. These include various preparatory steps (i.e., prior to tanning), such as soaking, fleshing and liming; and subsequent tanning, which includes several sub-processes and several post-tanning processes (including dyeing and coating). During these process steps animal skins and hides are processed in batches mixing hides from several sources, starting from the salting in the slaughterhouse through the various processes in the tannery and production line. Consequently, all information regarding the origin of the hide including the farm in which the animal was grown and the specific slaughterhouse from which the hide originated is lost. Furthermore, as hide moves along the production process and later as the finished leather advances through the production supply chains of leather products, valuable information regarding the origin of the material, the production process (e.g. the various processes it underwent, dates, batch numbers) as well as the supply chain-may be lost.

Tanneries and leather production facilities mark the hide or batches of hides by using various external tags and markings. However, there is a need for an inherent marking of the hide which is inseparable from the hide itself. Furthermore, there is a need to mark the animal skins and hides such that information regarding the origin of the hide (such as the farm, the area where the animal was grown, and/or the slaughter house) and additional information as may be needed, is encoded in an inseparable manner in the material itself. This information may be read at various stations along the production line and supply chains, allowing for better management of production processes and the supply chain. Such a marking system of hides and leather products may be also used for purposes of authentication, verification, and brand protection.

U.S. Patent Application No. 2019/0360992 [1] discloses a leather inspection apparatus for detecting inconsistencies on both the upper and lower surfaces of a hide. It includes a first camera assembly movably coupled to a support frame and capable of movement along the upper surface of the hide and a second camera assembly movably coupled to the support frame and capable of movement along the lower surface of the hide. A computing device is coupled to the first camera assembly and the second camera assembly, such that the first camera assembly detects the locations of inconsistencies in the upper surface of the hide and the second camera assembly detects the locations of inconsistencies in the lower surface of the hide; and a marking carriage movably coupled to the frame and operatively coupled to the computing device, wherein the marking carriage provides on the upper surface of the leather hide a visual indication of both the locations of the inconsistencies of the upper surface of the leather hide and the locations of the inconsistencies of the lower surface of the leather hide.

U.S. Pat. No. 9,951,394 [2] discloses methods and systems for processing raw animal hides into leather wherein a tanning manager directs the routing of hides to a plurality of tanning drums, allowing the tanning manager to use efficiently the capacity of the plurality of tanning drums and provides improved consistency in the tanning of the hides; enabling the tanning manager to route hides to tanning drums based on various factors, such as hide type, hide weight, and hide size; providing the tanning manager with greater control over the processing of hides; and providing a measure of traceability throughout the various tanning processes.

### BACKGROUND ART

[1] US Patent Application No. 2019/0360992
[2] U.S. Pat. No. 9,951,394

### General Description

The process of leather manufacturing from hides has changed relatively little over the years, with the many steps involved remaining substantially unchanged. A typical process comprises preliminary steps to preserve the hides and prepare them for tanning, followed by steps of tanning and subsequent further processing. The typical sequence of steps in the preliminary, pre-tanning stage of the process involves curing with salts, soaking and washing, unhairing, fleshing, splitting, deliming, bating and pickling.

Salt curing is achieved by immersing the hides in a brine solution, by wet or dry salting. An alternative curing process involves processing through the preliminary steps in the conventional manner and tanning with chromium sulphate, thus resulting in a product termed wet blue, which is thereafter tanned.

The inventors of the technology disclosed herein have developed a process for marking animal skins, hides and leathers which involves applying a formulation comprising one or more XRF-identifiable marker to hides or processed leathers, at any stage of the processing scheme mentioned above, under conditions that do not introduce any change to the leather processing steps (and thus do not have any effect on the resulting processed leathers), and also which securely and irreversibly embed the markers within the leather, thus enabling detection of the marker at any stage thereafter, including in a finished marketable leather product.

The markers may be detected and their concentration measured using a suitable reader throughout the production process of the leather and the final product made therefrom. The ability to introduce a marker at any stage of the process such that its presence may be detected at any stage thereafter, renders the marking technique of the invention especially unique for encoding into the hide latent information such as the origin of the hide (that is, the farm or the slaughterhouse from which the hide originated), various dates of production (e.g. the date of tanning), the processing facilities (tannery), the supplier or distributer of the hide, the grade of the hide and so on. Thus, by having the ability to mark or encode a hide or a processed leather at an early stage, and at any stage thereafter, the production history may be latently embedded within the final product.

As known in the art, “hide” refers to the integument or natural covering of an animal. The term is sometimes associated with larger animals. In the context of the invention disclosed herein the term refers to an animal skin of an animal of any size, including cattle, sheep, goats, pigs, buffalo, crocodiles, alligators, reptiles, etc. Leather is any collagen-containing material, with and without hair, which has been obtained by a tanning process, as disclosed herein, or by any leather processing scheme. The leather is thus any processed leather, furs and hides produced from animal skins, as known in the art. The leather may be of any animal source, of any size and thickness, and may be processed for making any leather-based products such as shoes, bags, clothing articles, furniture and others.

The marking of the hide or leather according to the present invention may be carried out at any stage along the production process of the leather. The steps involved in leather production which may be modified to comprise a step of marking according to the invention are generally the following:

In the first step, the animal skins are washed with soap and detergent to prevent microbial growth and subsequently unhaired by immersing the skins in an alkali solution that breaks down the hairs and leaves the skins with little or no hairs. The remaining hairs are removed in a step of liming, wherein a hairless skin is immersed in alkali and sulfide solutions to complete the removal of hair and further to alter the properties of the skin collagen. In the process, the collagen also becomes chemically modified and, as a result, swells, leaving open structures.

In the next step, deliming and bating takes place. In this step, the skin structure is further opened by treatment with enzymes. Unwanted materials that come off the skins are removed. Pickling involves treating the skins with an acid for prolonging their storage and preserving them for several years.

Tanning may be the most important and chemically complex step in leather production. During tanning, the skin structure is stabilized in its open form by replacing or associating some of the collagen with complex ions of chromium. Alternative green tanning procedures may also be utilized. Depending on the tanning procedure used, the color and texture of the leather may change. When leather has been tanned it is capable of withstanding harsh conditions such as boiling water.

Neutralizing, dyeing and fat liquoring refer to treatments with alkali solutions aimed to neutralize the tanned skins and prevent their deterioration. Subsequently, the leather may be dyed and treated with reactive oils that attach to the fibrous structure, thereby improving leather suppleness and flexibility. Drying and finishing proceeds to provide processed, stable and colored leather.

It should be noted that the processing stages detailed herein may vary. Processes of the invention are independent of any change in a process or step involved in a process for manufacturing leather from hide.

Thus, in a process for manufacturing leather from hide, the process comprising treating an animal skin, unprocessed hide or processed leather with a formulation comprising at least one XRF-identifiable marker under conditions permitting embedding said marker in the animal skin, unprocessed hide or processed leather. The process for the manufacturing of the leather comprises soaking of the skin, liming, deliming, bating, pickling and/or tanning, and the hide or leather so processed is treated with the formulation comprising the marker.

The invention also provides a process for identifying a leather, the process comprising treating an animal skin, unprocessed hide or processed leather with a formulation comprising at least one XRF-identifiable marker under conditions permitting embedding said marker in the animal skin, unprocessed hide or processed leather; and analyzing the presence of the XRF-identifiable marker in said animal skin, unprocessed hide or processed leather, to thereby identify the leather. The analysis may be carried out as disclosed herein.

The XRF-identifiable marker is selected to identify a particular property or information relating to the processed hide or leather and thus may thereafter be unequivocally identified and monitored. Where the hide or leather is treated more than once with different marker formulations, as defined herein, each of the marker formulations may provide a latent marking that identifies a different property or information. Additionally, the concentration of the marker can also be measured enabling encoding of information by associating different codewords for different concentrations of markers. Generally speaking, in leather production process, marking may be used to identify any one or more of the following:
origin of the leather;
the farm where the animal was grown;
the slaughterhouse;
the processing facility;
date of processing;
the processing protocol;
type of leather, e.g., quality and type of animal;
date of tanning;
the tannery;
the supplier of the processed hides; and
other information relating to operational and logistic data such as batch, year, week, factory, operator, and marketing data, including customer, distributer, collection, and information relating to the supply chain of hide, leather and leather product etc.

By enabling such latent encoding, identification and monitoring of the leather at any stage of processing, and even in a finished leather product, becomes possible.

The technique of the present invention may be used for managing and supervising the chains of production and supply of hides, leather, and leather products. The marking on the hide may be used to identify the origin of a hide or leather; namely, the farm in which the animal was grown, the manufacturer, various suppliers and distributors. The technique also enables classification of the hides or leather according to one or more selected parameters such as production site, year, etc.

A system for managing a supply chain of hides and leather may include a database system (central or distributed) where data relating to hides and their marking is stored. For example, the database system may contain information relating to the origin of the hide, the manufacturer of the leather produced from the hide, a batch of hides, leather, leather products as well as the future destinations (e.g. distributors and buyers). For that purpose, the device reading the marking (e.g. an XRF analyzer) may communicate with the database system. The database system may be an on-the-premises, cloud-based system or a distributed ledger. In an example, the database system may be a distributed blockchain system wherein a plurality of parties store and access relevant data. In such a blockchain system a plurality of parties (for example, parties which are members of the same supply chain) may store and access data wherein the data stored is immutable, easily verifiable and, due the distributed design, inherently resistant to modification. In an example, the parties to the blockchain system may include farms, tanneries and production facilities, suppliers, delivery companies, and even end users.

For example, the marking on the hide, leather and leather product is read (detected) by a suitable XRF device and recorded every time it changes hands along the supply chain and recorded (e.g. automatically) on the blockchain allowing each party to easily verify the provenance and complete history of the hide and/or leather. Blockchain systems that are suitable for managing a supply chain of marked objects and products are described in International Patent Applications PCT/IL2018/050499 and PCT/IL2019/050283 or any US applications derived therefrom, which are incorporated herein by reference.

The invention further provides a process for identifying a production and/or commercial history of a leather, the process comprising
treating an animal skin or an unprocessed hide with a formulation comprising a first XRF-identifiable marker at a first stage prior to tanning of said animal skin or unprocessed hide, to embed said first marker in the animal skin or the unprocessed hide; wherein the first marker encoding at least one first information set, e.g., relating to animal skin or unprocessed hide or process relating thereto;
following tanning of the unprocessed hide, treating the tanned hide with a second XRF-identifiable marker to embed said second marker in the tanned hide; wherein the second marker encoding at least one second information set, e.g., relating to tanning stage; and
analyzing the presence of the first and/or second XRF-identifiable markers in said tanned hide or in a product manufactured therefrom to identify information encoded in the first and/or second information set.

Generally speaking, the conditions used to embed the marker in the skin, hide or leather are those used in leather processing steps. No special conditions are utilized. This supports the uniqueness of processes of the invention whereby none of the processing conditions need to be modified to allow suitable and effective marking of the leather.

In another process the hide is marked before processing, during processing before tanning and during or after tanning. The process may thus comprise
treating an unprocessed animal skin or an unprocessed hide (possibly after salting or during salting) with a formulation comprising a first set of one or more XRF-identifiable markers to embed said first marker in the animal skin or the unprocessed hide; wherein the first marker encoding at least one parameter relating to the animal skin or unprocessed hide or process relating thereto (for example, the first set of markers may encode the farm from which the hide originated, the slaughterhouse, the breed of the animal, and the grade/quality of the hide and so on);
during processing before tanning (e.g. the preparatory stages) treating the hide with a formulation comprising a second set of one or more XRF-identifiable markers marker to embed said second set of markers in the hide; wherein the second set of markers encoding at least one parameter relating to the tanning stage, the grade of the hide after undergoing preparatory processing before tanning, batch number and so on;
during or after tanning (e.g. during dyeing), treating the tanned hide with a formulation comprising a third set of one or more XRF-identifiable markers to embed said third set of markers in the tanned hide; wherein the third set of markers encoding at least one parameter relating to post-tanning processing, for example, the grade of the hide after tanning, parameters relating to the dyeing process, the destination of the dyed leather and so no;
optionally including one or more other marking steps at any stage of the process; and
analyzing the presence of the first and/or second and/or third and/or optionally further sets of XRF-identifiable markers in said tanned hide or in a product manufactured therefrom to determine any of the parameters encoded.

By treating the skin, hide or processed leather, with a marker formulation, the marker becomes embedded or chemically associated or trapped within the skin, hide or processed leather to produce a substantially irreversible interaction with the marker. Markers which are applied to unprocessed (possibly salted) hide can be detected after preparatory stages of production (i.e. all production processes prior to tanning) and after the hide has undergone tanning. The markers can be detected on the hide after the hide has undergone dyeing and coating (finishing). The markers can also be read from the finished leather after production, and even from a final leather product.

In some embodiments, the animal skin or hide are treated at a stage prior to tanning. In some embodiments, the skin or hide are treated at the slaughterhouse or at the tannery. Marking that is applied to the hide at this initial stage was determined resilient enough to withstand the various often aggressive processes of leather production.

In some embodiments, the marking is applied to the hide during production in the preparatory stages of production prior to tanning. In other embodiments, the marking is applied to the hide during tanning.

In further embodiments, the marking is applied to the hide during dyeing and/or during the finishing processes.

In some embodiments, the marking is applied to the hide in the slaughterhouse prior to or during salting the hide, or after the salting process.

As demonstrated herein, the marking step may be fixed within, prior to or after any processing step of the hides and leathers without imposing any changes to any of the processing steps. Not only that the acceptable leather processing steps need not be modified in any way, the marking does not harm the hide, nor the leather made therefrom. A person versed in the art would not be able to differentiate a marked hide or a marked leather from one that is not marked according to the invention, unless examined by XRF.

Any of the marking steps involve treating an animal skin, unprocessed hide or processed leather with a formulation comprising at least one XRF-identifiable marker under conditions permitting embedding said marker in the animal skin, unprocessed hide or processed leather. The term “treating” or any lingual variation thereof involves contacting the sheet of hide with the formulation, by way of continuous washing or spraying with or soaking in a water-based formulation or other non-aqueous solutions, which includes one or more markers, herein the “marker formulation”. The marking steps may be preceded by an optional step of pre-soaking in which chemicals used in a prior leather processing step are washed off. For example, an unprocessed or salted hide may be soaked in a solution of water and soap in order to remove unwanted skin components and possibly salt from the hide. In the subsequent stage, the marking is carried out by treating, e.g., by soaking, the hide in a marker formulation. In some embodiments, the hide may be soaked in a marking formulation over a period of 30 minutes to 12 hours. The soaking may be done in a stirring vessel. Depending on a variety of factors, such as the particular hide, the stage of the processing etc, soaking the hide for an extended time during production may be impractical or inconvenient. For the purpose of speeding up the marking process, the process may be carried out in an ultrasonic bath, thus reducing the marking stage to a period of a few minutes.

Apart from the marker molecules or marker elements, the marker formulation may also include processing agents such as surfactants, catalysts and enzymes; and intermediate or bridging agents that are capable of chemically associating the marker to a region, a material or an atom of the treated hide or leather.

A sheet of skin or hide may be treated once with a marker formulation or may be treated with two or more marker formulations, at different stages of leather production process, wherein each of the two or more marker formulations may contain the same or different markers. By enabling consecutive marking sessions, each of the sheets of skins may be encoded with a variety of important information relating to origins, date of processing, site of processing etc. For example, a first marking session is performed on an unprocessed hide before the preparatory stage of production and then again during tanning and/or during the dyeing or the finishing stages of leather production. Surprisingly, notwithstanding the stage of application, the marker which is applied to the hide, even at the initial stage prior to production, remain embedded in or on the hide throughout the production process and may be read even from the final finished product.

The marker or marking formulation comprises at least one XRF-identifiable marker. The marker may be detected and measured by X-Ray fluorescence (XRF) spectrometers (readers) which detect and identify the marker response (signature) signal(s). The XRF readers may be Energy Dispersive X-Ray fluorescence EDXRF spectrometers. XRF markers are flexible, namely, they may be combined, blended or form compounds with a huge range of carriers and materials.

The marker can be water soluble, permitting facile and effective marker penetration into the hide or leather. However, where the marker is water-insoluble, the aqueous formulation may comprise the marker in suspended or dispersed forms. The marker may be in a form of a metal atom, a metal oxide, or a metal salt such as a metal sulfide, a metal carbonate, metal sulfate, metal carbide and others; or in the form of an organometallic or an organohalide material. The organometallic material may be selected amongst organic anions that are ionically associated with at least one metal atom (metal cation). Non-limiting examples include metal phenolates, metal acrylates, metal-associated anilines and others. The organohalide is at least one organic material substituted with at least one halide e.g., bromide, iodine, chloride. Such organohalides include halide-substituted phenols, halide substituted anilines, halide-substituted epoxies, halide-substituted acrylates, halide-substituted amides, halide-substituted acids, halide-substituted glycols and others.

Notwithstanding the type of marker, the marker is an atom or a material that is not present in the silk fibers; nor in any of the processing solutions typically used in silk production. Using a marker that is not native to the leather or the process for its processing, as disclosed herein, enables accurate and confident encoding and further generating a complex encoding scheme. Atoms or materials which may be present in the leather or involved in its manufacturing process and which may be regarded XRF-identifiable due to their composition or nature, contribute nothing to the ability contemplated herein to determine the production and commercial history of a silk-based product as such native or accidental materials do not constitute a code for determining history. The code relies only on a material added in a predefined concentration, composition and optionally in combination with one or more additional marker (atom or material). Thus, as used herein, the XRF-identifiable marker is one which is present in a marker formulation and is actively added or used for the purposes disclosed herein.

Thus, depending on the process or a particular step of the process, excluded are materials comprising metals used in typical processes of the art. Such materials may be metals salts or complexes or organic materials of metals such as chromium, aluminum and zirconium. Putting it differently, in processes or process steps utilizing aluminum metals to treat the hide, e.g., in a step of tanning, the marker material will not be or will not comprise aluminum. Same applies to chromium and zirconium.

Generally speaking, the metal or atom used as a marker may be any atom of the periodic table. The atom may be presented as a salt, a complex, an organic compound or an inorganic compound. For example, where the marker is a metal or a metal containing material, e.g., organometallic material, or metal salt the metal atom may be selected from aluminum (provided as e.g., aluminum sulfate), titanium (provided as, e.g., titanium sulfate), cobalt (provided as e.g., cobalt nitrate hexahydrate, cobalt gluconate hydrate, cobalt glycinate), nickel (provided as nickel nitrate hydrate, nickel glycinate), yttrium provided as e.g., yttrium nitrate hexahydrate), cadmium (provided as e.g., cadmium nitrate tetrahydrate), tin (provided as e.g., tin chloride), scandium, niobium, silver, tungsten, zinc, zirconium, manganese, copper, lead, molybdenum, vanadium, bismuth, antimony, tantalum and cesium (provided as e.g., cesium carbonate).

Other metal-based markers may be provided in a water-insoluble form. Such include aluminum oxide, scandium acetate, titanium oxide, cobalt acetyl acetonate, cobalt carbonate, cobalt dibromo, nickel acetyl acetonate, nickel acrylate, yttrium oxide, niobium oxide, silver carbonate, silver chloride, tin ethyl hexanoate, tungsten oxide and others.

Halide-based markers include tri-iodine phenol (TIP), tribromophenol (TBP), tri chlorophenol (TCP), 2,2-bis(bromomethyl) propane-1,3-diol, 2,4,6-tribromo aniline, pentabromobenzyl acrylate, 4,5,6,7-tetrabromoisobenzofuran-1,3-dione, ammonium bromide and others.

As stated herein, the marker formulation that is brought into contact with the hide or leather, or which the hide or leather is soaked in, comprises the marker and also a variety of processing agents such as surfactants, catalysts and enzymes; and intermediate or bridging agents which are capable of chemically associating the marker to a region, a material or an atom of the treated hide or leather. The processing agents may include ionic or nonionic, silicone-based or polymeric surfactants, having hydrophilic and hydrophobic groups, enzyme surfactants (generating enzymatic activity), and hydrogen peroxide. The hydrogen peroxide may be used to activate functional groups in the collagen present in the hide or leather as the main structural protein, thus facilitating better attachment of the marker to the hide or leather. The processing agents may include the following brands families: Peltec, Supralan, Trupowet, Max uni, Max 1, Max T2, Borron A, Truponat, Oropon, Pellvit, Actazym, Atlox, Hypermer, Proviera, ProSoak, Feliderm, Bemanol, Aglutan, Silastol, Derugan, Pristolamin, Basozym, Trupowet, and others.

Metal ions may be associated directly with functionalities on the collagen that are capable of such association. However, where the marker cannot directly associate to the collagen, a bridging molecule may be needed. Thus, the intermediate or bridging molecules are selected for attaching or bonding the marker atom or marker material to the hide. Without wishing to be bound by theory or a mode of operation, the intermediate molecules are bifunctional molecules that comprise at least two functionalities, capable of associating to the active residues in amino acids making up the collagen in the hide and also to the marking elements or molecules. The intermediate molecules may bond with a carboxylic, hydroxylic or an amine active residue present in any of the collagen amino acids. For example, an intermediate molecule may be an aldehyde such as an aromatic aldehyde. Similarly, the intermediate molecule may be chitosan, lignin, glutaraldehyde and its derivatives, N-hydroxysuccinimide (NETS), 1-ethyl-3-(3-dimethylaminopropyl)carbodiimide (EDC), or EDTA and derivatives thereof.

Other intermediate molecules may be such which contain sulfide groups, mercaptan groups, epoxides, imine groups, imide groups and other functionalities capable of reacting with a collagen functionality, e.g., carboxylic, hydroxylic or an amine active residue present in any of the collagen amino acids.

A reading unit may be used for detecting the marking compositions and/or measuring the concentrations or relative concentration of the markers in the preselected areas or complete area on the surface of the leather or processed hide. In an example the marking composition includes markers which are identifiable by XRF analysis and the verification unit comprises an XRF analyzer which emits an X-ray or Gamma-ray radiation towards the object and detects the X-ray signal (a response signal) that is emitted from the markers in response. Such an XRF analyzer may be configured to measure/estimate the concentration or relative concentration of each of the markers according to the detected response signal. The concentrations of the markers may be indicative of the information encoded by the marking composition on the object. Accordingly, based on the measured/estimated concentration the system may be configured and operable to verifying that the applied marker composition indeed matches/encodes the intended information/authentication data that should have had being marked on the object and possibly also verifies the quality of the marking applied by the marking device (i.e. the quality may be determined based on the signal to noise (SNR) of the detected signal.

### DETAILED DESCRIPTION OF EMBODIMENTS

### Example 1: Formulations According to the Invention

Various hides and processed lathers have been treated with a marking formulation according to the present invention. Non-limiting examples of such treatment protocols are listed in Table 1 below.

Shown in Table 1 are the marker molecule containing a marker atom or element, the hide exposure time (Mix. Time A), the mixing method (Mix Method B), the second exposure time (Mix Time B) and whether or not additional treatment steps have been included. These may be treatment with an ionic surfactant, a non-ionic surfactant, an enzymatic surfactant, lignin, chitosan, EDC, NHS or hydrogen peroxide.

TABLE 1
formulations according to embodiments of the present invention.

| | Mix. | Mix. | Mix. | | Non | | | | | | |
|---|---|---|---|---|---|---|---|---|---|---|---|
| | Time | Method | Time | Ionic | ionic | Enzymatic | | | | | |
| Marker molc. | A | B | B | surfactant | surfactant | surfactant | Lignin | Chitosam | EDC | NHS | H₂O₂ |
| VOSO4•xH2O | 15 min. | US | 2 min. | Yes | Yes | Yes | No | No | No | No | Yes |
| bis-Br-ethyl- | 15 min. | US | 2 min. | Yes | Yes | Yes | No | No | No | No | Yes |
| propanediol | | | | | | | | | | | |
| Co(NO3)2•6H2O | 15 min. | US | 2 min. | Yes | Yes | Yes | No | No | No | No | Yes |
| Co(NO3)2•6H3O | 15 min. | US | 2 min. | No | Yes | Yes | No | Yes | No | No | Yes |
| COAcAc | 15 min. | US | 2 min | Yes | Yes | Yes | No | No | No | No | Yes |
| CoCO3 | 15 min. | US | 2 min. | Yes | Yes | Yes | No | No | No | No | Yes |
| CsCO3 | 15 min. | US | 2 min. | No | Yes | Yes | No | Yes | No | No | Yes |
| Na28eO4 | 30 min. | US | 2 min. | Yes | Yes | Yes | No | No | No | No | No |
| Nb2O5 | 15 min. | US | 2 min. | Yes | Yes | Yes | No | No | No | No | Yes |
| NH4Br | 15 min. | US | 2 min. | Yes | Yes | Yes | No | No | No | No | No |
| Ni(NO3)2*6H2O | 1 h | US | 2 min. | Yes | Yes | Yes | No | No | No | No | Yes |
| Ni(NO3)2*6H2O | 30 min. | US | 2 min. | Yes | Yes | Yes | No | No | No | No | No |
| Ni(NO3)2*6H2O | 15 min. | US | 2 min. | No | Yes | Yes | No | Yes | No | No | Yes |
| NiAcAc | 15 min. | US | 2 min. | Yes | Yes | Yes | Yes | No | No | No | No |
| NiAcAc | 15 min. | US | 2 min. | Yes | Yes | Yes | Yes | No | No | No | Yes |
| NiAcAc | 15 min. | US | 2 min. | Yes | Yes | Yes | No | No | No | No | Yes |
| Sc(CH3CO2)3 | 1 h | US | 2 min. | Yes | Yes | Yes | No | No | No | No | Yes |
| Sn Ethyl | 15 min | US | 2 min. | Yes | Yes | Yes | Yes | No | No | No | Yes |
| Hexanoate | | | | | | | | | | | |
| SnCl2 | 15 min. | US | 2 min. | No | Yes | Yes | No | Yes | No | No | Yes |
| SnCl2*2H2O | 1 h | US | 2 min. | Yes | Yes | Yes | No | No | No | No | Yes |
| Tetrabromobenzo- | 15 min. | US | 2 min. | Yes | Yes | Yes | No | No | No | No | Yes |
| furandione | | | | | | | | | | | |
| Tribromoaniline | 15 min. | US | 2 min. | Yes | Yes | Yes | No | No | No | No | Yes |
| Tribromoaniline | 15 min. | US | 2 min. | No | Yes | Yes | No | No | Yes | No | No |
| Tribromoaniline | 15 min. | US | 2 min. | No | Yes | Yes | No | No | No | Yes | No |
| Trichloroaniline | 15 min. | US | 2 min. | No | Yes | Yes | No | No | No | Yes | No |
| Trichloroaniline | 15 min. | US | 2 min. | No | Yes | Yes | No | No | No | No | Yes |
| WO2 | 15 min. | US | 2 min. | Yes | Yes | Yes | Yes | No | No | No | Yes |
| Y(NO3)3*6H2O | 1 h | US | 2 min. | Yes | Yes | Yes | No | No | No | No | Yes |
| Y(NO3)3*6H2O | 15 min. | US | 2 min. | Yes | Yes | Yes | No | No | No | No | No |
| Y(NO3)3*6H2O | 15 min. | US | 2 min. | No | Yes | Yes | No | Yes | No | No | Yes |

In additional examples, the exemplary markers listed in Table 1 where mixed in the presence of an ionic surfactant in water or an aqueous formulation over a period of several minutes to several hours to embed the marker in the hide or processes leather, in various processing steps.

### Example 2

A 10 gr piece of hide was soaked in a solution of 30 gr water and 0.01-0.07 gr of anionic surfactant at a temperature of 25° C. for a duration of 15 minutes. The hide was then washed by soaking it in clean water for 5 minutes. In the next step the hide was soaked in a solution of 30 gr of buffer solution including 0.03-0.2 gr of a marking composition comprising, 0.02-0.08 gr of anionic surfactant, 0.01-0.08 gr enzyme, 0.01-0.08 gr of non-ionic surfactant in an ultrasonic stirrer, fora duration of 2 minutes, after which 0.01-0.04 gr of hydrogen peroxide were added to the solution and stirred for further 1 minute in the ultrasonic stirrer.

The hide was then examined by an XRF analyzer and the presence of the marking composition was detected.

Different hide samples were marked at each of the other leather processing steps and the presence of the marker was established subsequently.

Following these marking processes the hide has further undergone common leather production processes of soaking, liming, deliming and tanning.

At the end of the entire process, namely after the soaking, liming, deliming, and tanning the hide was examined by XRF analyzer and the presence of tha marker was detected.

### Example 3

A 10 gr piece of hide was soaked in a solution of 30 gr of water and 0.01-0.07 gr of anionic surfactant minutes at a temperature of 25° C. for a duration of 15 min. The hide was then washed by soaking it in clean water for 5 minutes. In the next step the hide was soaked in a solution of 30 gr of buffer solution including 0.01-0.5 gr of marking composition comprising WO₂, 0.01-0.08 gr of anionic surfactant, 0.01-0.08 gr of enzyme, 0.01-0.08 gr of non-ionic surfactant, 0.01-0.08 gr of bridging agent (lignin), in an ultrasonic stirrer, for a duration of 2 minutes, after which 0.01-0.04 gr of hydrogen peroxide were added to the solution and stirred for further 1 minute in the ultrasonic stirrer. The hide was then washed by soaking it in clean water for 5 minutes. In the next steps the hide underwent regular soaking, liming, deliming and tanning processes similar to ones described in example 1. At the end of the process after soaking, liming, deliming, and tanning the hide was examined by an XRF analyzer. The presence of the marking composition was detected.

### Example 4

A 10 gr piece of hide was soaked in a solution of 30 gr of water and 0.01-0.07 gr of anionic surfactant at a temperature of 25° C. for a duration of 15 minutes. The hide was then washed by soaking it in clean water for 5 minutes. In the next step the hide was soaked in a solution of 30 gr of buffer solution including 0.02-0.1 gr of marking composition comprising trichloroaniline, 0.01-0.08 gr enzyme, 0.01-0.08 gr of non-ionic surfactant, 0.01-0.08 gr of enzyme, and 0.04 gr of 0.01-0.05 NHS (N-Hydroxy Succinimide) in an ultrasonic stirrer, for a duration of 2 minutes, after which 0.03 gr of hydrogen peroxide were added to the solution and stirred for further 1 minute in the ultrasonic stirrer. The hide was then washed by soaking it in clean water for 5 minutes. In the next steps the hide underwent regular soaking, liming, deliming and tanning processes similar to ones described in example 1. After soaking, liming, deliming, and tanning the hide was examined by an XRF analyzer. The presence of the marking composition was detected.

### Example 5

A 10 gr piece of hide was soaked in a solution of 30 gr of water and 0.01-0.07 gr of anionic surfactant at a temperature of 25° C. for a duration of 15 minutes. The hide was then washed by soaking it in clean water for 5 minutes. In the next step the hide was soaked in a solution including 20 gr of buffer solution, 0.05-0.3 gr of marking composition comprising Y(NO₃)₃, 5-20 gr of Chitosan, 0.01-0.08 gr enzyme, 0.01-0.08 gr of non-ionic surfactant, 0.01-0.05 gr of H₂O₂ in an ultrasonic stirrer, for a duration of 2 minutes, after which NaOH 1M solution were added to set the solution a PH into level of 6 and stirred for further 2 minutes in the ultrasonic stirrer. The hide was then washed by soaking it in clean water for 5 minutes. In the next steps the hide underwent regular soaking, liming, deliming and tanning processes similar to the ones described in example 1. After soaking, liming, deliming, and tanning the hide was examined by an XRF analyzer. The presence of the marking composition was detected.

### Example 6

A 10 gr piece of hide was soaked in a solution of 30 gr of water and 0.01-0.07 gr of anionic surfactant at a temperature of 25° C. for a duration of 15 minutes. The hide was then washed by soaking it in clean water for 5 minutes. In the next step the hide was soaked in a solution including 20 gr of buffer solution, 0.05-0.3 gr of marking composition comprising SnCl₂, 5-20 gr of Chitosan, 0.01-0.08 gr enzyme, 0.01-0.08 gr of non-ionic surfactant, 0.01-0.05 gr of H₂O₂ in an ultrasonic stirrer, for a duration of 2 minutes, after which NaOH 1M solution were added to set the solution a PH into level of 6 and stirred for further 2 minutes in the ultrasonic stirrer. The hide was then washed by soaking it in clean water for 5 minutes. In the next steps the hide underwent regular soaking, liming, deliming and tanning processes similar to the ones described in example 1. After soaking, liming, deliming, and tanning the hide was examined by an XRF analyzer. The presence of the marking composition was detected.

### Example 7

A 10 gr piece of hide was treated according to the production steps until the tanning phase. After the pickling step, the markers were added in the drum at a concentration between 0.0025% and 0.06% and mixed for 20 minutes. The pH was adjusted to between 3.2 and 3.5 with an acid used in the pickling step (formic acid, sulfuric acid or others). Tanning was carried out by adding into the drum 6.5% chromium salts, 0.025%-0.30% of biocide or detergent. The tanning reaction can comprise also other additives such as wetting agents 0.8%, fixing agent 0.65%-0.70%, or slipping agents 0.15%. The reaction was mixed overnight at temperature between 27-30° C. at a pH between 2.8 and 3.0. The hide was successively washed by soaking with water and 0.05% of degreasing agent for 10 minutes and then in solution of water and 0.05% biocide for 20 minutes. The hide was placed on a paper towel to absorb the excess of water and examined by an XRF analyzer. The presence of the marking composition was detected.

1-33. (canceled)

34. A process for marking leather manufactured from hide with an XRF-identifiable marker, the process comprising treating a hide or a processed leather with a formulation comprising at least one XRF-identifiable marker to embed said marker in the hide or processed leather, to thereby obtain a marked hide or marked leather, wherein the XRF-identifiable marker is not a native material to a hide or leather or involved in a process for its manufacturing.

35. A process for identifying a production and/or commercial history of a leather or a product made therefrom, the leather or product having been marked with at least one XRF-identifiable marker, the process comprising directing an X-ray or Gamma-ray radiation towards the leather or product made therefrom and detecting a response X-ray signal emitted from the marker in response, such that said response signal is indicative of presence, concentration or relative concentration of the marker to thereby provide information encoded by the marker on the production or commercial history of the leather or product made therefrom.

36. The process according to claim 34, wherein the hide or processed leather is treated more than once with different marker formulations, each of the marker formulations provides a latent marking identifying a different property or information.

37. The process according to claim 36, wherein the latent marking identifies any one or more of origin of the hide or leather, a slaughterhouse, a processing facility, date of processing, processing protocol, type of leather, quality of leather, type of animal, date of tanning, tannery, supplier of the hides, operational information and marketing information.

38. The process according to claim 35, wherein the production and/or commercial history comprises origin of hide or leather, a slaughterhouse, a processing facility, date of processing, processing protocol, type of leather, quality of leather, type of animal, date of tanning, tannery, supplier of the hides, operational information and marketing information.

39. The process according to claim 34, wherein the marker formulation is applied to the hide during production in a preparatory stage of leather production prior to tanning.

40. The process according to claim 34, wherein the marker formulation is applied to the hide during tanning.

41. The process according to claim 34, wherein the marking is applied to the hide during dyeing and/or during the finishing processes.

42. The process according to claim 34, the marker formulation is applied to the hide in the slaughterhouse prior to or during salting the hide, or after the salting process.

43. The process according to claim 34, wherein treating comprises continuous washing or spraying with the marker formulation or soaking in a water-based marker formulation or solution.

44. The process according to claim 34, comprising pre-soaking of the hide or processed leather.

45. The process according to claim 34, wherein the treating of the hide or processed leather is carried out in an ultrasonic bath.

46. The process according to claim 34, wherein the marker formulation comprises a processing agent and an intermediate or bridging agent.

47. The process according to claim 46, wherein the processing agent is selected from surfactants, catalysts and enzymes.

48. The process according to claim 46, wherein the intermediate or bridging agent is selected to chemically associate the marker to a region, a material or an atom of the hide or processed leather.

49. The process according to claim 48, wherein the intermediate or bridging agent is a bifunctional molecule comprising at least two functionalities, capable of associating to active residues in amino acids making up collagen in the hide and to the XRF identifiable marker.

50. The process according to claim 49, wherein the intermediate or bridging agent is an aldehyde containing molecule, sulfide containing molecules, mercaptan containing molecules, epoxide containing molecules, imine containing molecules, imide containing molecules, chitosan, lignin, glutaraldehyde or EDTA.

51. The process according to claim 34, the process comprising two or more marking sessions, each session comprising treating the hide or leather with a marker formulation comprising same or different XRF identifiable marker.

52. The process according to 50, wherein the two or more marking sessions are carried out at different stages of the leather production process.

53. The process according to claim 52, wherein a first session is carried out on a hide before a preparatory stage of production and a further session during tanning and/or during dyeing or a finishing stage of leather production.

54. A process for identifying a production and/or commercial history of a leather, the process comprising
treating a hide with a formulation comprising a first XRF-identifiable marker at a first stage prior to tanning of said hide, to embed said first marker in the hide;
following tanning of the hide, treating the tanned hide with a second XRF-identifiable marker to embed said second marker in the tanned hide; and
analyzing the presence of the first and/or second XRF-identifiable markers in said tanned hide or in a product manufactured therefrom.

55. The process according to claim 54, wherein the analyzing step comprises directing an X-ray or Gamma-ray radiation towards the leather or product made therefrom and detecting a response X-ray signal emitted from the marker in response, such that said response signal is indicative of presence, concentration or relative concentration of the marker to thereby provide information encoded by the marker on the production or commercial history of the leather or product made therefrom.
